SEQUENCE LISTING (8 sequences)

SEQ ID NO: 1 - RNA, 13 nt, synthetic construct
  misc_feature 1..13: note = reRNA
  source 1..13: mol_type = other RNA
aatggcaatggca

SEQ ID NO: 2 - RNA, 13 nt, synthetic construct
  misc_feature 1..13: note = ssRNA
  source 1..13: mol_type = other RNA
aatggcaatggca

SEQ ID NO: 3 - RNA, 64 nt, synthetic construct
  misc_feature 1..64: note = crRNA_ORF gene
  source 1..64: mol_type = other RNA
gatttagactaccccaaaaacgaaggggactaaaacccaacctcttctgtaatttttaaactat

SEQ ID NO: 4 - RNA, 64 nt, synthetic construct
  misc_feature 1..64: note = crRNA_S gene
  source 1..64: mol_type = other RNA
gatttagactaccccaaaaacgaaggggactaaaacgcagcaccagctgtccaacctgaagaag

SEQ ID NO: 5 - RNA, 159 nt, SARS-CoV-2
  source 1..159: mol_type = viral cRNA
tgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctag

SEQ ID NO: 6 - RNA, 177 nt, SARS-CoV-2
  source 1..177: mol_type = viral cRNA
taacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgc

SEQ ID NO: 7 - DNA, 177 bp, synthetic construct
  misc_feature 1..177: note = cDNA_ORF gene for SARS-CoV-2 RNA synthesis
  source 1..177: mol_type = other DNA
taatacgactcactataggatctattaagtaatccaaagaattatcattctgatcttaacagatgtattcgtcggtaatctagacacaccggttggagaagacattaaaaatttgataataaacgaccaaattcatattacagaggatgttgaagccatcaaaagtgtaatgtgagt

SEQ ID NO: 8 - DNA, 195 bp, synthetic construct
  misc_feature 1..195: note = cDNA_S gene for SARS-CoV-2 RNA synthesis
  source 1..195: mol_type = other DNA
taatacgactcactatagcgtgtcagatgtcgtagacattaccaaggtaaaagtaatataaaattatcttttcaggatccaacttctattgggtgtattattcgacgtcgtggtcgacaggttggacttcttcttagtggtcctcagtttattgaagatacatttcgttcatttcaaactttggatcactacaat

(19) United States
(12) Patent Application Publication
(10) Pub. No.: US 2023/0280301 A1
(43) Pub. Date: Sep. 7, 2023

(54) ELECTROCHEMICAL BIOSENSOR FOR DETECTING TARGET RNA

(71) Applicant: Industry-Academic Cooperation Foundation, Yonsei University, Seoul (KR)

(72) Inventors: Hyo Il JUNG, Seoul (KR); Woong HEO, Seoul (KR); Kyung Yeon LEE, Seoul (KR)

(73) Assignee: Industry-Academic Cooperation Foundation, Yonsei University, Seoul (KR)

(21) Appl. No.: 18/056,693

(22) Filed: Nov. 17, 2022

(30) Foreign Application Priority Data
Nov. 18, 2021 (KR) 10-2021-0159837
May 27, 2022 (KR) 10-2022-0065162

(51) Int. Cl. G01N 27/327; G01N 27/30; B82Y 15/00

(52) U.S. Cl. CPC G01N 27/3278; G01N 27/308; B82Y 15/00

(57) ABSTRACT

The present invention relates to an electrochemical biosensor for detecting a target RNA, and the present invention can detect a very small amount of target RNA with high sensitivity without a nucleic acid amplification reaction through a CRISPR/Cas13a trans-cleavage reaction, thereby having an advantage of being useful for point-of-care diagnostic testing of fast-spreading RNA-based infectious diseases such as COVID-19.

### CROSS REFERENCE TO RELATED APPLICATION

The present application claims priority to Korean Patent Application No. 10-2021-0159837, filed Nov. 18, 2021, and Korean Patent Application No. 10-2022-0065162, filed May 27, 2022, the entire contents of which are incorporated herein for all purposes by this reference.

### INCORPORATION BY REFERENCE OF SEQUENCE LISTING

The present application is being filed along with a Sequence Listing in electronic format. The Sequence Listing is provided as a file entitled 717572007200SeqList.xml, created Nov. 13, 2022, which is 10,904 bytes in size. The information in the electronic format of the Sequence Listing is incorporated by reference in its entirety.

### BACKGROUND OF THE INVENTION

### Field of the Invention

The present invention relates to an electrochemical biosensor for detecting a target RNA, and more particularly, to an electrochemical biosensor for detecting a target RNA, which can detect an RNA such as SARS-CoV-2 with high sensitivity through a CRISPR/Cas13a trans-cleavage reaction without a nucleic acid amplification reaction.

### Description of the Related Art

The COVID-19 pandemic caused by severe acute respiratory syndrome coronavirus 2 (SARS-CoV-2) has seriously threatened the health and economy of the world's population. According to the World Health Organization (WHO), by Aug. 2, 2021, 205 million people have been infected and 4 million have died due to COVID-19. The virus spread rapidly from person to person through various routes such as direct contact, air, medium, and droplets, and SARS-CoV-2 has an infection reproduction number R₀=3.1, which is highly contagious compared to Middle East respiratory syndrome coronavirus (MERS-CoV) (R₀=0.6), severe acute respiratory syndrome coronavirus (SARS-CoV) (R₀=0.7), and influenza virus (R₀=1.3). A pandemic situation like this can be managed by screening suspected cases of COVID-19 through rapid diagnosis of viral infection and isolating infected patients to suppress further spread of virus.

Meanwhile, a real-time reverse transcription polymerase chain reaction (RT-PCR) technology is currently the standard technology for detecting a SARS-CoV-2 RNA with high sensitivity and specificity, but the applicability of RT-PCR in point-of-care testing (POCT) is limited because RT-PCR technology requires trained personnel and takes a long test time (approximately 3 to 4 hours) including sample preparation and gene amplification. Minimizing test time is key for rapid point-of-care diagnosis. The immunodetection method has emerged as an alternative to the SARS-CoV-2 monitoring method due to its potential to obtain test results quickly, but false negatives due to low accuracy and low sensitivity may rather exacerbate the spread of SARS-CoV-2 (Cui, et al., 2020; Huang, et al., 2021).

Clustered regular interspaced short palindromic repeats (CRISPR)/CRISPR-associated (Cas) protein systems targeting specific nucleotide sequences are attracting attention as highly effective detection and rapid monitoring strategies. The trans-cleavage activity of Cas13a and Cas12a in CRISPR systems applied to biosensors can be used for non-specific single-stranded RNA and DNA functionalized with fluorescent dyes and quencher-tag reporter molecules. For example, Gootenberg, et al. was able to detect viral RNA with 1.0×10⁻¹ fg/ml through fluorescence signal analysis using CRISPR technology integrated with recombinase polymerase amplification (RPA), which is a nucleic acid amplification technology to detect Zika virus RNA (Gootenberg, et al., 2017; Broughton, et al., 2020; Wang, et al., 2020). However, despite the superior detection limits and specificity provided by CRISPR-based optical detection strategies, their bulky and expensive optics may limit their utility in point-of-care test applications. On the other hand, the electrochemical measurement technique can be used as an alternative method for quantifying a small amount of target gene due to its high sensitivity, specificity, miniaturization, portability, and cost-effectiveness. These advantages of electrochemical detection have led to the development of an electrochemical biosensor combined with the CRISPR/Cas12a system to detect human papillomavirus (HPV) and parvovirus at concentration of 2.8×10⁶ fg/ml and 6.0×10² fg/ml, respectively, without nucleic acid amplification (Zhang, et al., 2020; Dai, et al., 2019). This method has the advantage that the detection procedure is simpler by omitting the pre-amplification step, but this biosensor shows relatively low performance in monitoring low concentrations of viral RNA, which is essential for early detection of viral infections, compared to conventional CRISPR/Cas-based sensing methods such as a specific high sensitivity enzymatic reporter unlocking (SHERLOCK) and DNA endonuclease targeted CRISPR trans reporter (DETECTR) (Kellner, et al., 2019; Chen, et al., 2018).

Accordingly, the present inventors made diligent efforts to overcome the limitations of the existing SARS-CoV-2 detection method and CRISPR/Cas-based method, and as a result, the inventors were able to develop an electrochemical biosensor that combined the trans-cleavage activity of the CRISPR/Cas13a system with an electrode on which a highly conductive nanostructure was deposited. The inventors completed the present invention by confirming that when the biosensor was used, it was possible to effectively detect COVID-19 with high-sensitivity analysis performance by the sensor performance that tracked a very small amount of SARS-CoV-2 RNA even without the pre-amplification step so that it was able to utilize a target RNA monitoring platform that was able to detect various target RNAs with high accuracy and sensitivity without nucleic acid amplification process.

### DOCUMENTS OF RELATED ART

(Non-Patent Document 1) Cui, et al., 2020. Biosensors and bioelectronics 165, 112349.
(Non-Patent Document 2) Huang, et al., 2021. Biosensors and Bioelectronics 171, 112685.
(Non-Patent Document 3) Gootenberg, J. S. et al., 2017. Science 356(6336), 438-442.
(Non-Patent Document 4) Broughton, J. P. et al., 2020. Nature biotechnology 38(7), 870-874.
(Non-Patent Document 5) Wang, M., Zhang, R., Li, J., 2020. Biosensors and Bioelectronics 112430.
(Non-Patent Document 6) Zhang, D. et al., 2020. ACS sensors 5(2), 557-562.
(Non-Patent Document 7) Dai, Y. et al., 2019. Angewandte Chemie 131(48), 17399-17405.
(Non-Patent Document 8) Kellner, M. J. et al., 2019. Nature protocols 14(10), 2986-3012.
(Non-Patent Document 9) Chen, J. S. et al., 2018. Science 360(6387), 436-439.

### SUMMARY OF THE INVENTION

An object of the present invention is to provide a biosensor, a method for manufacturing a biosensor, a method for detecting a target RNA, and a kit for detecting a target RNA, which can detect a very small amount of target RNAs with high sensitivity analysis performance without a nucleic acid amplification reaction.

In order to achieve the above object, the present invention provides a biosensor for detecting a target RNA in which a reporter RNA (reRAN) is immobilized on an electrode on which a nanocomposite (NC) containing molybdenum disulfide (MoS₂), graphene, and chitosan (CHT) and a flower-shaped gold nanostructure (AuNF) are deposited.

In the present invention, the biosensor may react with a Cas13a-crRNA-target RNA complex so that a current is reduced.

In the present invention, the biosensor may be coated with a blocking agent.

In the present invention, the blocking agent may be BSA, SKIM MILK, SALMON SPERM DNA, or mercaptohexanol (MCH).

In the present invention, the reporter RNA may be immobilized on the electrode on which the nanocomposite and nanostructure are deposited through a streptavidin-biotin bond, an avidin-biotin bond, or a thiol-gold bond

In the present invention, the nanocomposite may contain the molybdenum disulfide, the graphene, and the chitosan in a volume ratio of 1:0.3 to 0.7:0.05 to 0.3.

In the present invention, the reporter RNA may be tagged with a redox molecule.

In the present invention, the redox molecule may be methylene blue, toluidine blue or ferrocene.

In the present invention, the electrode may be a carbon electrode.

The present invention also provides a method for manufacturing the biosensor, including the steps of:
(a) sequentially depositing the nanocomposite containing the molybdenum disulfide (MoS₂), the graphene, and the chitosan and the flower-shaped gold nanostructure on the electrode; and
(b) immobilizing the reporter RNA (reRNA) tagged with a redox molecule to the electrode on which the nanocomposite and nanostructure are deposited.

In the step (a) of the present invention, 3-mercaptopropionic acid (MPA) may be treated on a carbon electrode on which the nanocomposite and the nanostructure are sequentially deposited.

In the step (a) of the present invention, 0.05 M to 0.5 M of the 3-mercaptopropionic acid may be treated for 10 minutes to 1 hour.

In the step (a) of the present invention, the 3-mercaptopropionic acid-treated carbon electrode may be coated with a blocking agent.

In the present invention, the blocking agent may be BSA, SKIM MILK, SALMON SPERM DNA, or mercaptohexanol (MCH).

In the step (a) of the present invention, the carbon electrode on which the nanocomposite and nanostructure are sequentially deposited may be coated with streptavidin, avidin, or biotin.

In the step (b) of the present invention, in order to interact with the streptavidin, avidin, or biotin coated on the gold on the carbon electrode surface or the electrode surface in the step (a), the reporter RNA (reRNA) each bound to a biotin group, a streptavidin group, or a thiol group may be reacted and immobilized.

In the step (a) of the present invention, 1 mg/ml to 20 mg/ml of the streptavidin may be added to be coated.

In the step (b) of the present invention, a reaction with 50 μg/ml to 500 μg/ml of the reporter RNA may be performed for 2 hours to 6 hours.

The present invention also provides a method for detecting a target RNA using the biosensor, including the steps of:
(a) treating a Cas13a-crRNA-RNA mixed sample on the biosensor; and
(b) measuring a reduced current amount of the biosensor.

In the present invention, the target RNA may be a SARS-CoV-2 RNA,

the crRNA may be the crRNA of an ORF gene represented by a nucleotide sequence of SEQ ID NO: 3 and/or a S gene represented by a nucleotide sequence of SEQ ID NO: 4.

In the present invention, in the Cas13a-crRNA-RNA mixture sample, an RNA sample may be additionally mixed with a mixture in which the Cas13a and the crRNA are mixed in a mass ratio of 1:0.1 to 0.001.

In the step (a) of the present invention, the Cas13a-crRNA-RNA mixture sample may be treated on the biosensor and reacted for 1 hour to 2 hours.

In the present invention, the RNA sample may be included in a biological sample selected from the group consisting of whole blood, plasma, serum, urine, saliva, runny nose, upper respiratory tract mucus, lower respiratory tract mucus, excretion, lymph, amniotic fluid, and tissue, or the RNA sample may be selected from the biological sample.

The present invention also provides a kit for detecting a target RNA including the provided biosensor, Cas13a, and a target RNA-specific crRNA.

In the present invention, the target RNA may be a SARS-CoV-2 RNA, and

the crRNA may be the crRNA of an ORF gene represented by a nucleotide sequence of SEQ ID NO: 3 and/or a S gene represented by a nucleotide sequence of SEQ ID NO: 4.

### BRIEF DESCRIPTION OF THE DRAWINGS

FIG. 1 illustrates a schematic diagram of an electrochemical biosensing strategy used in conjunction with CRISPR/Cas13a for SARS-CoV-2 detection according to an embodiment of the present invention. Virus RNAs are extracted from saliva collected from infected patients using a lysis buffer and mixed with a solution containing a Cas13a-crRNA complex. This complex binds to the SARS-CoV-2 RNA and triggers enzymatic activity. The activated Cas13a-crRNA complex is then loaded onto the sensor surface to cleave the reRNA immobilized on the electrode. The presence of SARS-CoV-2 can be quantified through the analysis of current change.

In FIG. 2, a) is a schematic diagram for manufacturing an electrochemical biosensor for SARS-CoV-2 RNA detection. The various modification steps of the biosensing surface were characterized by b) CV and c) EIS for (i) AuNF/NC/SPCE, (ii) SA/AuNF/NC/SPCE, (iii) BSA/SA/AuNF/NC/SPCE, (iv) reRNA/BSA/SA/AuNF/NC/SPCE, (v) cleaved reRNA/BSA/SA/AuNF/NC/SPCE. d) represents the current change obtained by DPV for steps (iv) and (v).

FIG. 3 illustrates a) an atomic force microscopy (AFM) micrograph and b) cross-sectional profile of AuNF/NC/SPCE, SA/AuNF/NC/SPCE, and BSA/SA/AuNF/NC/SPCE, and c) a confocal microscopy images of BSA/SA/AuNF/NC/SPCE, reRNA/BSA/SA/AuNF/NC/SPCE, and cleaved reRNA/BSA/SA/AuNF/NC/SPCE.

FIG. 4 illustrates a gel electrophoresis result performed to determine a) a ratio of crRNA in various concentrations of Cas13a in the formation of Cas13a-crRNA complex and b) the trans-cleavage performance of SARS-CoV-2 RNA by activated Cas13a-crRNA complex, and c) a schematic diagram of Cas13a-crRNA-based fluorescence analysis. Fluorescence intensity was examined with various concentrations of Cas13a-crRNA complexes at fixed concentrations of d) S gene and e) ORF gene.

FIG. 5 illustrates a result confirming the capture efficiency of a) crRNA_ORF gene and b) crRNA_S gene by Cas13a at various concentrations.

FIG. 6 illustrates in a) and b) a result confirming the incubation time of 3-mercaptopropionic acid (MPA).

FIG. 7 illustrates a result of confirming the concentration of streptavidin (SA) coated on an electrode surface.

FIG. 8 illustrates a result confirming the immobilization of reRNA at different a) concentrations and b) time.

FIG. 9 illustrates an evaluation of the appropriate time for trans-cleavage activity.

FIG. 10 illustrates a) DPV response of reRNA/BSA/SA/AuNF/NC/SPCE when various concentrations [a: 1.0×10⁻¹, b: 1.0×10⁰, c: 1.0×10¹, d: 1.0×10², e: 1.0×10³, f: 1.0×10⁴, and g: 1.0×10⁵ fg/ [symbol] ] of ORF genes are present in 0.1M PBS at pH 7.4 containing 0.1M KCl solution, b) a corresponding calibration curve of the sensor, and c) DPV response of reRNA/BSA/SA/AuNF/NC/SPCE when various concentrations [a: 1.0×10⁻¹, b: 1.0×10⁰, c: 1.0×10¹, d: 1.0×10², e: 1.0×10³, f: 1.0×10⁴, and g: 1.0×10⁵ fg/ml] of S gene are present in 0.1M PBS at pH 7.4 containing 0.1M KCl solution, and d) a corresponding calibration curve of the sensor.

### DETAILED DESCRIPTION OF THE INVENTION

Unless defined otherwise, all technical and scientific terms used herein have the same meaning as commonly understood by one of ordinary skill in the art to which this invention belongs Generally, the nomenclature used herein are those well-known and commonly employed in the art.

In the present invention, an electrochemical biosensor capable of quantifying an extremely low concentration of SARS-CoV-2 RNA without a nucleic acid amplification step was developed. The electrode of the biosensor is modified with a nanocomposite (NC) and a flower-shaped gold nanostructure (AuNF) to increase the conductivity of the electrode and the surface-to-volume ratio of the working electrode. Reporter RNA (reRNA) molecules tagged with methylene blue (MB) and biotin at each end were fixed to the electrode coated with streptavidin (SA).

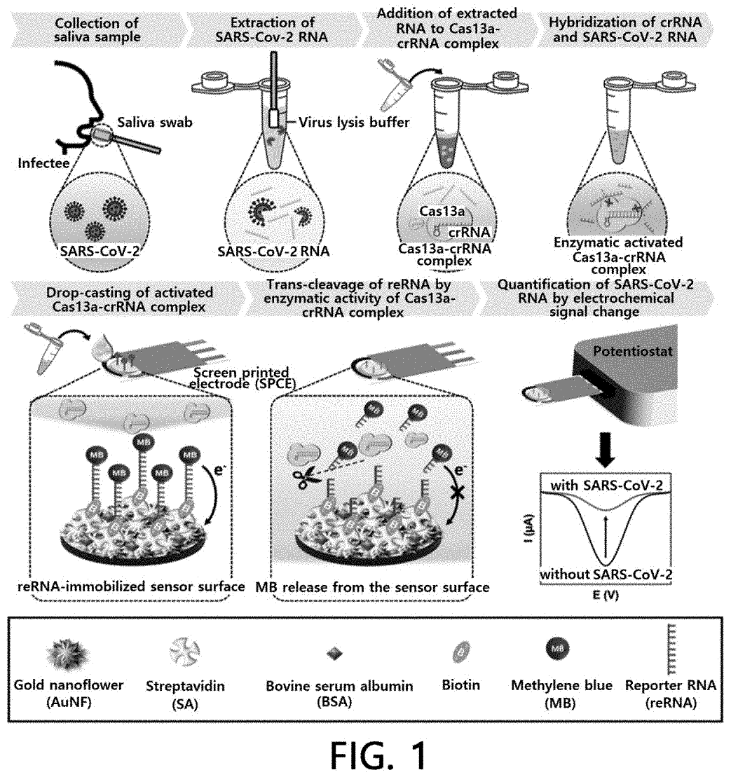

A sensing strategy for detecting the SARS-CoV-2 based on trans-cleavage of Cas13a-mediated reporter RNA using the modified electrode according to the present invention is illustrated in FIG. 1. First, saliva is collected with a cotton swab, viral RNA is extracted using a lysis buffer, and the extracted viral RNA is loaded onto the Cas13a-crRNA complex solution. This complex can recognize a specific sequence of SARS-CoV-2 RNA based on the crRNA sequence that complementarily binds to the target site of a target viral RNA via a proto spacer-flanking site (PFS) (Bruch, et al., 2021. Biosensors and Bioelectronics 177, 112887). When the Cas13a-crRNA complex is activated by binding to a specific region of SARS-CoV-2 RNA, non-specific cleavage of non-specific single-stranded RNA (ssRNA) is induced (van Dongen, et al., 2020. Biosensors and Bioelectronics 166, 112445; Zuo et al., 2017. Nature Biomedical Engineering 1(6), 1-2). The Cas13a-crRNA-SARS-CoV-2 RNA ternary complex is then introduced to the sensor surface, and methylene blue (MB) is released from the biosensing surface as the reporter RNA immobilized on the sensor is concomitantly cleaved into short fragments. This in turn eliminates electron transfer from a redox probe to the electrode surface, reducing a peak current. Finally, the SARS-CoV-2 RNA is quantified by transducing the reduced peak current. The developed biosensor was able to detect the ORF and S genes of SARS-CoV-2 at low levels of 4.4×10⁻² fg/ml and 8.1×10⁻² fg/ml, respectively. The sensor's performance to track a small amount of SARS-CoV-2 RNA showed great promise as a monitoring platform for COVID-19 diagnosis with high-sensitivity assay performance even by omitting the pre-amplification step.

Accordingly, in one aspect, the present invention relates to a biosensor for detecting a target RNA in which a reporter RNA (reRAN) is immobilized on an electrode on which a nanocomposite (NC) containing molybdenum disulfide (MoS₂), graphene, and chitosan (CHT) and a flower-shaped gold nanostructure (AuNF) are deposited.

In the present invention, the biosensor may react with a Cas13a-crRNA-target RNA complex so that a current is reduced.

In the present invention, the biosensor may be coated with a blocking agent.

In the present invention, the blocking agent may be BSA, SKIM MILK, SALMON SPERM DNA, or mercaptohexanol (MCH), but is not limited thereto.

In the present invention, the reporter RNA may be immobilized on the electrode on which the nanocomposite and nanostructure are deposited through a streptavidin-biotin bond, an avidin-biotin bond, or a thiol-gold bond, but is not limited thereto. That is, any pair of compounds that bind to each other in the art may be applied to the reporter RNA and the electrode without limitation.

In the present invention, in order for the reporter RNA to be immobilized on the electrode, the reporter RNA may be bound to streptavidin, avidin, biotin, or thiol.

In one aspect, the electrode on which the nanocomposite and nanostructure are deposited may be bound to biotin so as to be bound to the streptavidin or avidin bound to the reporter RNA.

In another aspect, the electrode on which the nanocomposite and the nanostructure are deposited may be bound to streptavidin or avidin so as to be bound to the biotin bound to the reporter RNA.

In another aspect, when a thiol group is bound to the reporter RNA, the reporter RNA may bind to gold deposited on the electrode.

In the present invention, the nanocomposite may contain molybdenum disulfide, graphene, and chitosan in a volume ratio of preferably 1:0.3 to 0.7:0.05 to 0.3, more preferably 1:0.4 to 0.6:0.08 to 0.15, most preferably about 1:0.5:0.1, but is not limited thereto.

In the present invention, the molybdenum disulfide in the form of molybdenum disulfide nanosheets (MoS₂ NS), and the graphene in the form of graphene nanoplatelets (GNP) may be contained in the nanocomposite, but the present invention is not limited thereto.

In the present invention, the nanosheet may function to increase the conductivity of the electrode, and the nanoplatelet may function to increase the conductivity and biocompatibility of the electrode.

In the present invention, the gold nanostructure may be preferably a flower-shaped gold nanostructure (AuNF) to increase biocompatibility and conductivity in the biosensor (see Sensors and Actuators: B. Chemical 357 (2022) 13), which has a larger surface area than a general gold nanostructure (AuNP), and thus has the advantage of allowing a wider range of signal measurement by fixing more reporters on the electrode surface.

In the present invention, the reporter RNA may be tagged with a redox molecule.

In the present invention, the redox molecule may be methylene blue, toluidine blue or ferrocene, but is not limited thereto.

In the present invention, the electrode may be a carbon electrode.

In the present invention, the reporter RNA reacts with a Cas13a-crRNA-target RNA complex and is cleaved to induce the escape of a redox molecule, such as a methylene blue molecule, from the electrode surface, and consequently to reduce the current. Compounds that reduce the current at the electrode surface can be used without limitation.

In another aspect, the present invention relates to a method for manufacturing the biosensor including the steps of:
(a) sequentially depositing the nanocomposite containing the molybdenum disulfide (MoS₂), the graphene, and the chitosan and the flower-shaped gold nanostructure on an electrode; and
(b) immobilizing the reporter RNA (reRNA) tagged with a redox molecule to the electrode on which the nanocomposite and nanostructure are deposited.

In the step (a) of the present invention, 3-mercaptopropionic acid (MPA) may be treated on a carbon electrode on which the nanocomposite and the nanostructure are sequentially deposited.

In the step (a) of the present invention, 0.05 M to 0.5 M of the 3-mercaptopropionic acid may be treated for 10 minutes to 1 hour.

In the step (a) of the present invention, the 3-mercaptopropionic acid-treated carbon electrode may be coated with a blocking agent.

In the present invention, the blocking agent may be BSA, SKIM MILK, SALMON SPERM DNA, or mercaptohexanol (MCH), but is not limited thereto.

In the step (a) of the present invention, a carbon electrode on which the nanocomposite and nanostructure are sequentially deposited may be coated with streptavidin, avidin, or biotin.

In the step (b) of the present invention, in order to interact with the streptavidin, avidin, or biotin coated on the gold on the carbon electrode surface or the electrode surface in the step (a), the reporter RNA (reRNA) each bound to a biotin group, a streptavidin group, or a thiol group may be reacted and immobilized.

In the step (a) of the present invention, 1 mg/ml to 20 mg/ml of the streptavidin may be added to be coated.

In the step (b) of the present invention, the reaction with 50 μg/ml to 500 μg/ml of the reporter RNA may be performed for 2 hours to 6 hours.

In the present invention, the electrode may be a carbon electrode.

As a preferred aspect of the present invention, the present invention may provide a method for manufacturing the biosensor including the following steps:
(a) sequentially depositing the nanocomposite (NC) containing a molybdenum disulfide nanosheet (MoS₂ NS), a graphene nanoplatelet (GNP), and the chitosan (CHT) and the flower-shaped gold nanostructure (AuNF) on a carbon electrode;
(b) treating 3-mercaptopropionic acid (MPA) on the carbon electrode on which the nanocomposite and the flower-shaped gold nanostructure are deposited;
(c) sequentially coating streptavidin and BSA on the carbon electrode treated with the 3-mercaptopropionic acid; and
(d) reacting biotinylated reporter RNA (reRNA) to the carbon electrode coated with streptavidin and BSA, and immobilizing the biotinylated reporter RNA on the electrode.

In the present invention, the molybdenum disulfide in the form of molybdenum disulfide nanosheet (MoS₂ NS), and the graphene in the form of graphene nanoplatelet (GNP) may be contained in the nanocomposite, but the present invention is not limited thereto.

In the step (b) of the present invention, preferably 0.05 M to 0.5 M, more preferably 0.08 M to 0.2 M, most preferably about 0.1 M of 3-mercaptopropionic acid may be treated for preferably 10 minutes to 1 hour, more preferably 20 minutes to 40 minutes, most preferably about 30 minutes, but the present invention is not limited thereto.

In the step (c) of the present invention, preferably 1 mg/ml to 20 mg/ml, more preferably 5 mg/ml to 15 mg/ml, most preferably 10 mg/ml of streptavidin may be added to be coated, but the present invention is not limited thereto.

In the step (d) of present invention, preferably 10 μg/ml to 500 μg/ml, more preferably 50 μg/ml to 200 μg/ml, most preferably about 100 μg/ml of the reporter RNA may be reacted for preferably 2 hours to 6 hours, more preferably 3 hours to 5 hours, and most preferably about 4 hours, but the present invention is not limited thereto.

In another aspect, the present invention relates to a method for detecting a target RNA using the biosensor, including the steps of:
(a) treating a Cas13a-crRNA-RNA mixture sample on the biosensor; and
(b) measuring a reduced current amount of the biosensor.

In the present invention, the target RNA may be a SARS-CoV-2 RNA, and the crRNA may be the crRNA of the ORF gene represented by the nucleotide sequence of SEQ ID NO: 3 and/or the S gene represented by the nucleotide sequence of SEQ ID NO: 4, but is not limited thereto.

In the step (a) of the present invention, the Cas13a-crRNA-RNA mixture sample may be treated on the biosensor and reacted for preferably 30 minutes to 3 hours, more preferably 1 hour to 2 hours, most preferably about 1 hour and 30 minutes, but the present invention is not limited thereto.

In the present invention, in the Cas13a-crRNA-RNA mixture sample of step (a), an RNA sample to be tested may be additionally mixed with a mixture in which the Cas13a and the crRNA are mixed in a mass ratio of 1:0.1 to 0.001.

In this case, the order in which the Cas13a, the crRNA, and the RNA sample are mixed may be arbitrarily changed and is not limited to a specific order.

In the present invention, the RNA sample may be included in a biological sample selected from the group consisting of whole blood, plasma, serum, urine, saliva, runny nose, upper respiratory tract mucus, lower respiratory tract mucus, excretion, lymph, amniotic fluid, and tissue, or the RNA sample may be selected from the biological sample, but the present invention is not limited thereto.

In the present invention, the RNA sample may be an RNA sample extracted by dissolving cells, microorganisms, or viruses with a lysis buffer, but is not limited thereto.

In the present invention, the target RNA in the RNA sample may be hybridized to the crRNA to form a triple complex of Cas13a-crRNA-target RNA.

In the present invention, the method for detecting a target RNA may include the step of (c) determining that the biological sample is a target RNA-positive sample when the current amount of the biosensor is reduced by 10% or more compared to the case of treating with the control not containing the target RNA.

In the present invention, the biosensor may detect the target RNA of about 1.0×10⁻¹ fg/ml.

In another aspect, the present invention relates to a kit for detecting a target RNA including the biosensor, Cas13a, and a target RNA-specific crRNA.

In the present invention, the target RNA may be a SARS-CoV-2 RNA, and the crRNA may be the crRNA of an ORF gene represented by a nucleotide sequence of SEQ ID NO: 3 and/or a S gene represented by a nucleotide sequence of SEQ ID NO: 4, but is not limited thereto.

In the present invention, the kit may include the Cas13a and the target RNA-specific crRNA in the form of a mixture, and the kit may further include an instruction for detecting the target RNA.

In the present invention, the kit may further include a lysis buffer for extracting the RNA sample from the biological sample.

In the present invention, the kit may further include a stick or cotton swab for separating the biological sample from the human body, and may further include a tube for temporarily storing the biological sample, such as an Eppendorf tube.

In order to suppress the rapid spread of SARS-CoV-2 infection, a point-of-care (POC)-based detection method that can overcome the shortcomings of existing methods such as low accuracy of immunodetection method and long test time of RT-PCR is required. In the present invention, the electrochemical biosensor is utilized together with the trans-cleavage activity of Cas13a to enable sensitive quantification of SARS-CoV-2 RNA without a nucleic acid amplification step. By sequentially depositing the nanocomposite and AuNF on the electrode surface, the conductivity was improved and the surface area was enlarged, thereby improving detection performance. In order to maximally enhance the signal of the sensor, the appropriate ratio of Cas13a and crRNA for complex formation and trans-cleavage activity from the capture of SARS-CoV-2 RNA was determined through gel electrophoresis and fluorescence intensity measurement, so that the optimal concentration of Cas13a-crRNA that was able to maximize the signal enhancement of the sensor was selected. The electrochemical sensing platform of the present invention was able to detect the ORF and S genes in a wide linear dynamic range of 1.0×10⁻¹ fg/ml to 1.0×10⁵ fg/ml with LODs of 4.4×10⁻² fg/ml and 8.1×10⁻² fg/ml, respectively. The quantification of the SARS-CoV-2 RNA at concentrations below fg/ml with an electrochemical sensor using the CRISPR/Cas13a was achieved for the first time.

In addition, the observed recovery values (96.54% to 101.21%) show good applicability in the salivary matrix of the sensor according to the present invention and offer the possibility to directly use saliva samples without RNA purification. The biosensor according to the present invention can be used as a monitoring platform for diagnosis of COVID-19 with very sensitive analytical performance without a pre-amplification step, and can be used for on-site detection of various nucleic acids for rapid and accurate screening of pathogenic diseases.

### EXAMPLES

Hereinafter, the present invention will be described in more detail through examples. These examples are only for illustrating the present invention in more detail, and it will be apparent to those skilled in the art that the scope of the present invention is not limited by these examples according to the gist of the present invention.

### Example 1. Experimental Method

1-1. Experimental Material and Apparatus

Graphene nanoplatelets (GNPs), chitosan (CHT), gold(III) chloride trihydrate, sodium chloride, 3-mercaptopropionic acid (MPA), 1-ethyl-3-(3-(dimethylamino) propyl) carbodiimide hydrochloride (EDC), N-hydroxysuccinimide (NHS), ethyl alcohol, and bovine serum albumin (BSA) were purchased from Sigma-Aldrich (USA). Tris-Borate-EDTA (TBE) buffer (10×), agarose, and UltraPure™1 M Tris-HCl Buffer were purchased from ThermoFisher (USA). Molybdenum disulfide nanosheets (MoS₂ NSs) was purchased from Graphene Supermarket (USA). DNA ladder solutions (25/100 base, 25/100 bp, [symbol] 1143 kb) and LoadingSTAR reagent were purchased from DyneBio (South Korea). CRISPR/Cas13a protein was purchased from MCLAB (USA). RNA Cleanup Kit and HiScribe™T7 High Yield RNA Synthesis Kit were purchased from Monarch®145 (USA) and New England BioLabs (England), respectively. DNA and RNA oligonucleotides (Table 1) used in the present invention were synthesized and purified by BIONEER (South Korea).

TABLE 1

| | | Seq ID |
|---|---|---|
| Name | Sequence (5′→3′) | NO. |
| reRNA | /Methylene blue/AAUGGCAAUGGCA/3Bio/ | 1 |
| SSRNA | /Cy5/AAUGGCAAUGGCA/3Bio/ | 2 |
| crRNA_ORF | GAUUUAGACUACCCCAAAAACGAAGGGGACUAAAACCCAACCUCU | 3 |
| gene | UCUGUAAUUUUUAAACUAU | |
| crRNA_S | GAUUUAGACUACCCCAAAAACGAAGGGGACUAAAACGCAGCACCA | 4 |
| gene | GCUGUCCAACCUGAAGAAG | |
| ORF gene | UGAGUGUAAUGUGAAAACUACCGAAGUUGUAGGAGACAUUAUACU | 5 |
| | UAAACCAGCAAAUAAUAGUUUAAAAAUUACAGAAGAGGUUGGCCA | |
| | CACAGAUCUAAUGGCUGCUUAUGUAGACAAUUCUAGUCUUACUAU | |
| | UAAGAAACCUAAUGAAUUAUCUAG | |
| S gene | UAACAUCACUAGGUUUCAAACUUUACUUGCUUUACAUAGAAGUUA | 6 |
| | UUUGACUCCUGGUGAUUCUUCUUCAGGUUGGACAGCUGGUGCUGC | |
| | AGCUUAUUAUGUGGGUUAUCUUCAACCUAGGACUUUUCUAUUAAA | |
| | AUAUAAUGAAAAUGGAACCAUUACAGAUGCUGUAGACUGUGC | |

Disposable screen-printed carbon electrodes (SPCE; C110) were purchased from Dropsens Inc. in Spain. Electrochemical experiments were performed using a CHI-650E electrochemical analyzer (CH Instruments, USA). Gel electrophoresis was performed using a Mupid-exU system (Takara, Japan), and subsequent analysis was performed using a MiniBIS UV-transilluminator (DNR Biolmaging Sytems, Israel). In addition, total RNA purity and concentration were determined using a Nanodrop 2000 spectrophotometer (Thermo Fisher Scientific, USA). Atomic Force Microscopy (AFM) imaging and surface roughness analysis were performed using an NX-10 apparatus (Park Systems, South Korea). Confocal microscopy image analysis was performed using an LSM 700 (Carl Zeiss, Germany). Fluorescence intensity was measured using a Nanodrop 3300 fluorospectrometer (Thermo Fisher Scientific, USA).

1-2. Preparation of AuNF/NC/SPCE

SPCE was washed with cyclic voltammetry (CV) technology over the range of 0.1V to 0.7V in 0.5M H₂SO₄ solution to obtain uniform voltammetry. NC was prepared by mixing MoS₂ NS, GNP, and CHT in a volume ratio (quantity) of 10:5:1, respectively (see Kashefi-Kheyrabadi, et al., Biosensors and Bioelectronics 169, 112622. 2020). Then, a fixed amount of the NC solution was drop-cast on the surface of the working electrode. After NC deposition, AuNF was formed in 10 mM HAuCl₄ solution using previously reported methods including an amperometric technique performed at 0.2 V for 600 seconds.

1-3. Biosensing Surface Modification

A sensing surface was prepared as follows. First, AuNF/NC/SPCE was treated with 0.1M MPA for 30 minutes to form a layer of carboxyl groups on the electrode surface. 7 μl of the mixture solution composed of 0.5 mg/ml SA and EDC/NHS (0.2M and 0.05, respectively) in 0.1M of 2-(N-morpholino)ethanesulfonic acid (MES) (pH 4.7) was incubated at 23° C. for 2 hours in the dark to induce an amide bond between the amine group of SA and the carboxyl group on the electrode surface. In order to inactivate non-specific adsorption of the remaining active sites, 7 μl of 0.01% BSA solution was treated on the electrode surface at 23° C. for 10 minutes. Finally, a reRNA mixture containing reRNA (40 μg/ml), RNase inhibitor (4 U/μl), and TRIS-HCl buffer solution (40 mM) was added to the electrode to immobilize the reRNA through SA-biotin binding. In each modification step, unbound materials were removed by rinsing with deionized water and 0.1M phosphate buffered saline (PBS) (pH 7.4).

1-4. Gel Electrophoresis

A suitable Cas13a:crRNA ratio for the formation of the Cas13a-crRNA complex was examined by 2% agarose electrophoresis. Before adding the Cas13a-crRNA mixture to the agarose gel, crRNA was stained with LoadingSTAR and incubated with Cas13a at 37° C. for 30 minutes. Electrophoresis was performed in 1×TBE buffer (pH 8.1) at 45 V for 20 minutes. The intensities of the lines were examined using ImageJ software and the capture efficiency of crRNA by Cas13a was calculated as follows.

$$Capture efficiency = \frac{\begin{matrix}Initial intensity of crRNA - \\ Intensity of unbound cas 13 a - crRNA complex\end{matrix}}{Initial intensity of crRNA} \times 100$$

Trans-cleavage activity was examined by analyzing the gel result of the SARS-CoV-2 RNA by the Cas13a-crRNA complex. The SARS-CoV-2 RNA and the crRNA were stained and then mixed with the Cas13a. After incubation at 37° C. for 30 minutes, the mixture was loaded onto the gel and developed with 1×TBE buffer solution (pH 8.1) at 45V for 20 minutes. The images of the two electrophoresis results were visualized with a MiniBIS UV-transilluminator.

1-5. Cas13a-crRNA-Based Fluorescence Analysis

The fluorescence intensity was measured after incubating 1 ng/ml of SARS-CoV-2 RNA, 10 μg/ml of ssDNA in which of each end was labeled with a fluorophore (6-carboxyfluorescein; FAM) and quencher (Iowa Black), and a mixture of Cas13a and crRNA at different concentrations, respectively, at 37° C. for 30 minutes. Fluorescence signals were measured using a NanoDrop 3300 fluorospectrometer.

1-6. Electrochemical Measurement

The biosensing surface was characterized by electrochemical impedance spectroscopy (EIS) at 5.0×10⁻³ M with CV and 0.10 M KCl at a scan rate of 0.1 Vs⁻¹. Nyquist plots were recorded at an open-circuit potential and an AC potential of 0.005 V in various frequency ranges between 10 kHz and 0.1 Hz. DPV was used to quantify the SARS-CoV-2 RNA in the range of −0.5 V to −0.1 V, and voltammograms were measured at a pulse amplitude of 0.025 V and a scan rate of 0.05 Vs⁻¹.

### Example 2. Sensing Surface Characterization

The manufacturing steps of the biosensor are illustrated in FIG. 2a. In the early stage of electrode modification, a nanocomposite (NC) composed of MoS₂NS, GNP and CHT was applied to a bare electrode to enhance electron transport in a sensor (see L. Kashefi-Kheyrabadi et al, Biosensors and Bioelectronics 169 (2020) 112622)).

AuNF was electrodeposited to increase electrode conductivity (Wang, et al., 2011. Biosensors and Bioelectronics 30(1), 151-157). Next, SA and BSA were sequentially coated to immobilize reRNA and block the activated sensor surface, respectively. Biotin and MB-labeled reRNA were immobilized on the electrode by SA-biotin binding. Finally, after capturing the SARS-CoV-2 RNA, the activated Cas13a-crRNA complex was added to the sensor to induce the release of a redox molecule (e.g., methylene blue). As a result, the current signal was decreased

The various modification steps on the sensor surface were characterized by CV and EIS ((b) and (c) in FIG. 2). AuNF/NC/SPCE showed two redox peaks with high background current, indicating high conductivity of improved sensor surface (curve (i) of (b) in FIG. 2). The lower Faraday peak current and more pronounced peak potential separation observed after SA immobilization on the surface showed that negatively charged reRNAs were accumulated on the sensor surface and electron transfer between the [Fe(CN)₆]³⁻ redox probe and the sensing surface was impeded (curve (ii) of (b) in FIG. 2). The peak current was further relaxed upon subsequent blocking of the surface with BSA to prevent non-specific adsorption (curve (iii) of (b) in FIG. 2). When reRNA was immobilized through SA-biotin binding on the sensing surface, a trend similar to the decrease in current was identified (curve (iv) of (b) in FIG. 2). However, when the Cas13a-crRNA-target RNA was loaded on the surface and the reRNA was cleaved, the current slightly increased due to the degradation of substances that prevented electron transfer to the electrode (curve (v) of (b) in FIG. 2). Characterization of the biosensing surface was also verified by EIS ((c) in FIG. 2). At the high frequency, no semicircle was observed in AuNF/NC/SPCE, indicating that the electron transfer resistance in the electrode was negligible (curve (i) of (c) in FIG. 2). As a result of sequentially immobilizing the SA and the BSA to the electrode surface, the layer formed on the surface repelled [Fe(CN)₆]^{3-/4-} redox molecules, increasing the electron transfer resistance (curves (ii) and (iii) of (c) in FIG. 2). By immobilizing the reRNA on the electrode surface, a stack for negative charge was distributed on the surface. The increase in electron transfer resistance was due to the significant repulsion of [Fe(CN)₆]^{3-/4-} redox molecules against the negatively charged surface (curve (iv) of (c) in FIG. 2). Finally, treatment of the sensing surface with the activated Cas13a-crRNA complex reduced the interfacial electron transport resistance by alleviating the obstacles associated with the access of [Fe(CN)₆]^{3-/4-} redox molecules to the biosensing surface (curve (v) of (c) in FIG. 2). The results of CV and EIS were consistent with each other, showing that the surface preparation for biosensing was satisfactorily performed, and the current change due to the trans-cleavage effect on reRNA was also characterized through the DPV signal ((d) in FIG. 2). Cleavage of the reRNA induced the escape of MB molecules from the electrode surface and consequently decreased the current.

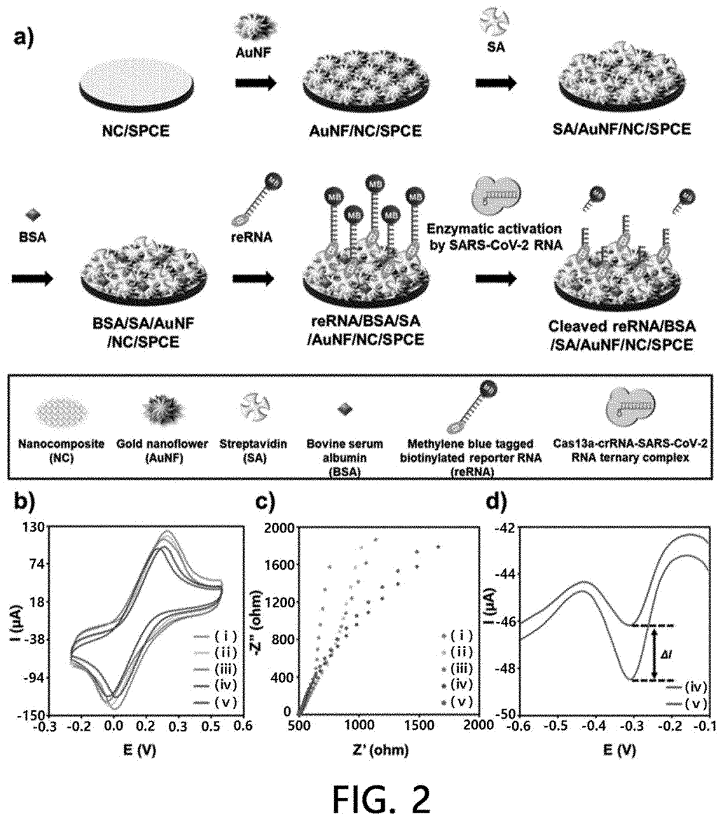

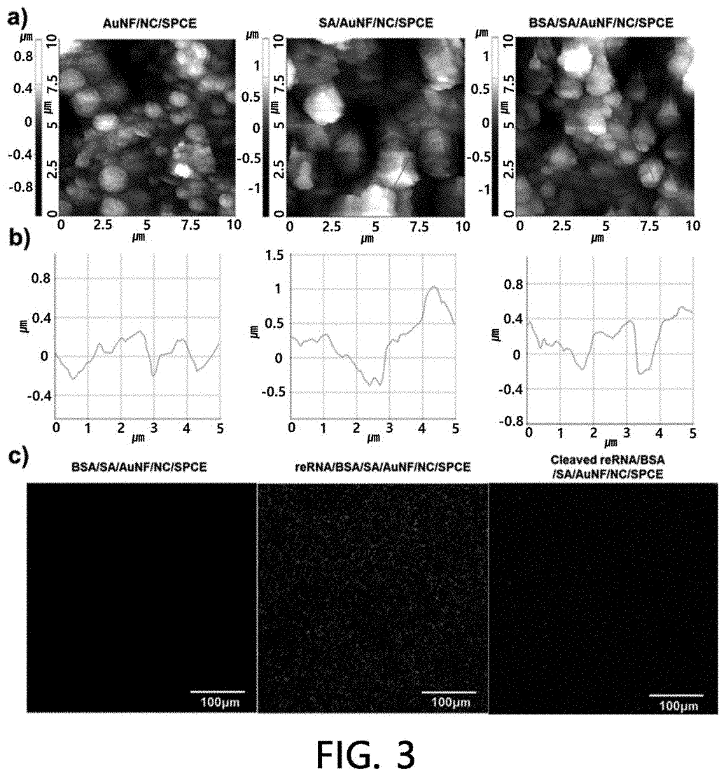

Roughness changes due to different modification steps were also defined through AFM measurements. AFM results with electrochemical results were compared at each modification step. In FIG. 3, (a) and (b) illustrate the measurement results of AFM micrographs obtained in each modification step until BSA blocking and cross-sectional descriptions accordingly. AuNF/NC/SPCE was confirmed to have rough topography with an average roughness (Rq) of 128.3±12.5 nm. Immobilization of SA to AuNF/NC/SPCE increased the observed Rq to 370.5±17.4 nm, indicating successful formation of the SA layer on the uniformly formed AuNF structure. The Rq value of BSA/SA/AuNF/NC/SPCE after surface blocking was 206.1±13.2 nm, lower than that of SA/AuNF/NC/SPCE, which may be because the gap between the immobilized SAs by BSA was filled. AFM measurements for reRNA/BSA/SA/AuNF/NC/SPCE and cleaved reRNA/BSA/SA/AuNF/NC/SPCE were also performed, but no significant change in roughness was observed due to the small size of reRNA (10 nt) and resolution in the non-contact mode of AFM measurements (data not illustrated) (Marrese, et al., 2017. J. Funct. Biomater, 8(1), 7). Therefore, reRNA immobilization and trans-cleavage activity were verified through image analysis using cyanine (Cy5) (fluorescent dye)-tagged reRNA. ReRNA was immobilized on BSA/SA/AuNF/NC/SPCE to induce fluorescence signals on the sensor surface (left and middle in (c) in FIG. 3). In contrast, after treatment with the activated Cas13a-crRNA complex bound to the target RNA, the fluorescence signal was decreased due to the trans-cleavage effect of Cas13a (right in (c) in FIG. 3). These results confirmed that the reRNA was actually immobilized (genuine immobilization) and that the reRNA was cleaved by Cas13a. Also, the AFM and fluorescence results were consistent with the electrochemical results of each modification step of the electrode surface.

### Example 3. Effect of Cas13a:crRNA Ratio on Trans-Cleavage of SARS-CoV-2 RNA

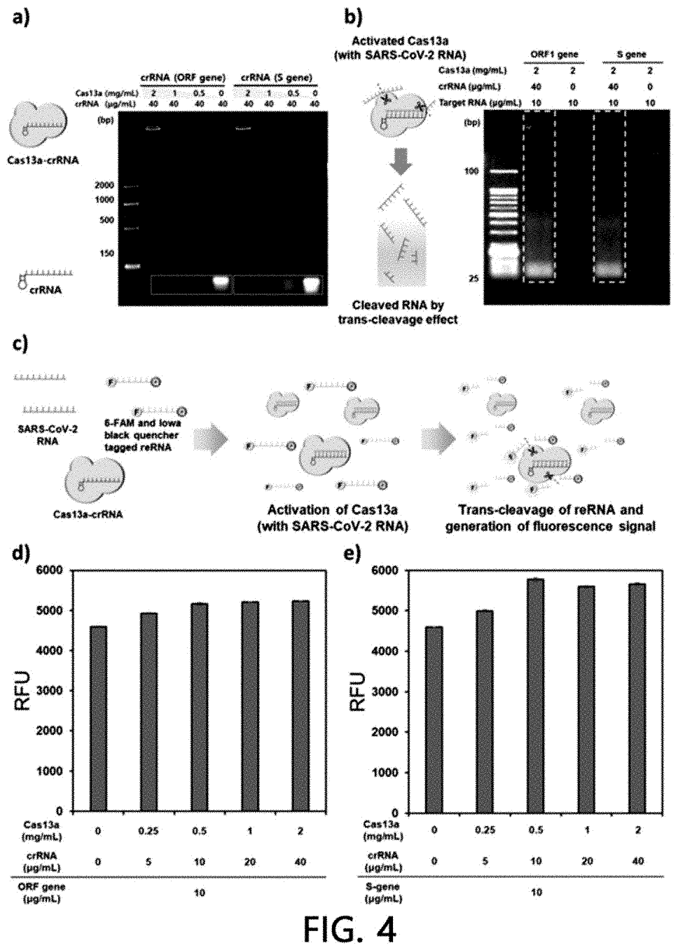

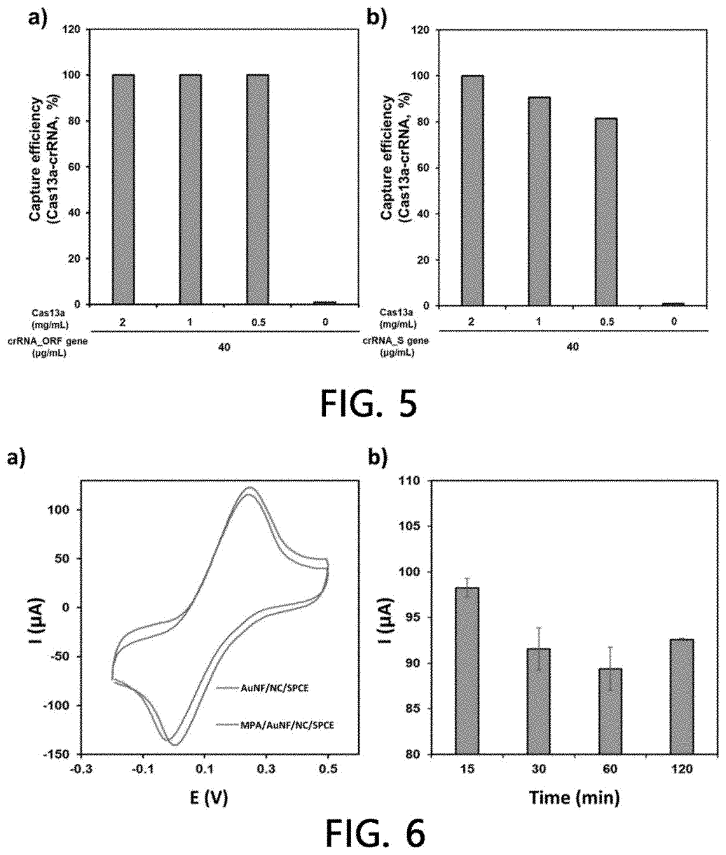

According to the CRISPR-based sensing mechanism, the cleavage function for ssRNA is activated by the combination of SARS-CoV-2 RNA and Cas13a-crRNA complex (Wang, et al., 2020, Biosensors and Bioelectronics 112430). To apply this CRISPR-based sensing mechanism to electrochemical measurements, it is essential to determine the ratio between Cas13a and crRNA to form the Cas13a-crRNA complex that can induce high trans-cleaving activity. To examine the formation of the complex, a mixture of crRNA and Cas13a protein that hybridizes with specific regions of the ORF and S sequences of SARS-CoV-2 was incubated and then gel electrophoresis was performed ((a) in FIG. 4). The previously reported SARS-CoV-2 sequence was used as a target sequence (Zhang, et al., 2020, A protocol for detection of COVID-19 using CRISPR diagnostics). Various concentrations of Cas13a protein (2-fold dilution ranging from 0.5 mg/ml to 2.0 mg/ml and 0 mg/ml control) were mixed with a single concentration of crRNA (40 μg/ml) and the mixed solution was added to the gel, and the intensity of the lower band was examined in the electrophoresis result. Weaker signal intensity indicates more effective binding of crRNA to Cas13a. In order to quantify the capture efficiency of crRNA for Cas13a, the strength of unbound Cas13a-crRNA was subtracted from the initial strength of crRNA and divided by the initial strength of crRNA through image J software analysis (FIG. 5). It was confirmed that the crRNA targeting the ORF gene (ORF gene_crRNA) was completely captured at a concentration of Cas13a of 0.5 mg/ml or higher, but the crRNA targeting the S gene (S gene_crRNA) had achieved 100% capture efficiency only at 2 mg/ml. Therefore, 2 mg/ml of Cas13a per 40 μg/ml of crRNA was chosen as the ratio for targeting these two sequences of SARS-CoV-2 RNA. Then, the presence or absence of a SARS-CoV-2 RNA cleavage reaction was evaluated using gel electrophoresis ((b) in FIG. 4). Each SARS-CoV-2 gene was mixed with the Cas13a-crRNA complex solution and incubated for 1 hour. As a result of electrophoresis, when the SARS-CoV-2 RNA was added to the Cas13-crRNA solution, a gradient was observed, which indicates that the trans-cleavage activity of Cas13a cleaves the remaining fragment of the SARS-CoV-2 RNA after cis-cleavage of a specific site of viral RNA. Finally, the concentration of Cas13a-crRNA complex to maximize the enzymatic function of Cas13a was examined using SHERLOCK's method with 6-FAM and Iowa black quencher-tagged ssRNA ((c) in FIG. 4) (de Puig, et al., 2021. Science Advances 7(32), eabh2944; Fozouni, et al., 2021. 2021. Cell 184(2), 323-333. e329). The RNase activity of Cas13a cleaves the ssRNA to increase the distance between the quencher and the fluorescent dye, inducing a fluorescence signal. The amounts of S gene, ORF gene, and ssRNA were fixed, and various concentrations of Cas13a and crRNA were mixed, followed by incubation at 37° C. for 2 hours. In both genes, the fluorescence signal gradually increased to 5 mg/ml of Cas13a and 10 μg/ml of crRNA and then saturated ((d), (e) in FIG. 4). Therefore, the appropriate concentrations of Cas13a and crRNA were estimated to be 0.5 mg/ml and 10 μg/ml, respectively.

### Example 4. Optimization of Detection Conditions

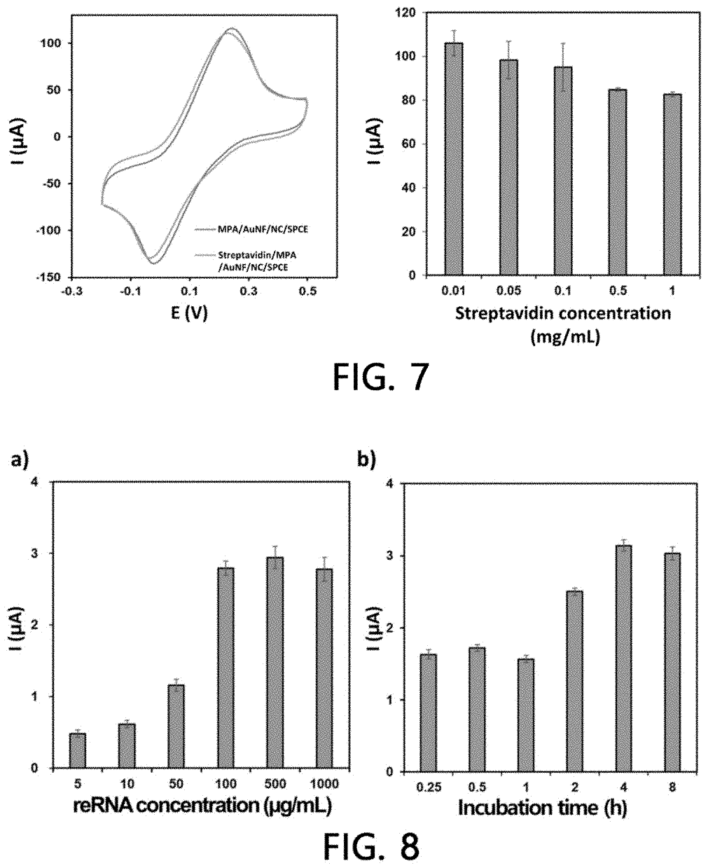

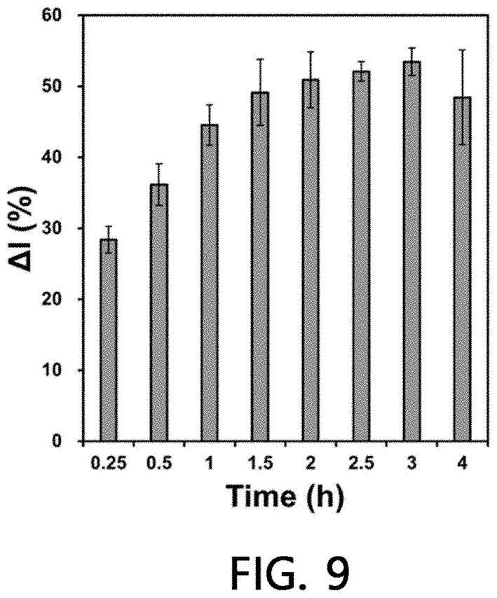

Experimental parameters such as MPA treatment time, streptavidin concentration, reRNA concentration, reRNA immobilization time, and trans-cleavage time were examined to achieve the optimal performance of the developed sensor. The incubation time of 0.1 M MPA to maximize the coated number of SAs was analyzed by CV. The lower peak current after MPA treatment indicates that electron transport was stopped by the formation of self-assembled monolayers ((a) in FIG. 6). Bipolar peak current was decreased by 30 minutes of incubation time, but there was no significant decrease in peak current after 30 minutes of MPA treatment ((b) in FIG. 6). Therefore, the incubation time of MPA was determined to be 30 minutes. In addition, the concentration of SA was examined to increase the number of reRNA immobilizations. In FIG. 7, (a) illustrates the cyclic voltammetry of the electrode surface before and after SA coating for 2 hours. A similar trend was observed, such as a decrease in current after SA coating, due to the disturbance of electron transport. The SA concentration was chosen to be 0.5 mg/ml because no decrease in the anode peak current was measured above 0.5 mg/ml of SA ((b) in FIG. 7). To evaluate the incubation time and concentration of reRNA, the optimal conditions were searched for by measuring the reduction signal of MB using DPV. The appropriate concentration of reRNA for immobilization on the BSA/SA/AuNF/NC/SPCE surface was analyzed by adding reRNA at different concentrations for 2 hours and comparing the signals. In FIG. 8, (a) illustrates that the DPV signal increases rapidly with increasing concentration of reRNA and is stabilized at 100 μg/ml of reRNA. This is probably due to complete immobilization of reRNA on BSA/SA/AuNF/NC/SPCE by reRNA. Therefore, it was determined that the reRNA concentration of 100 μg/ml was suitable for sensor preparation. The reRNA immobilization time (0.25 hours to 8 hours) was sequentially analyzed ((b) in FIG. 8). The reduction current increased as the immobilization time progressed and stabilized over 4 hours. As a result, 4 hours were applied as an appropriate time for reRNA immobilization on the electrode surface. In addition, the trans-cleavage time representing the biosensor with optimal analysis performance was examined (FIG. 9). reRNA/BSA/SA/AuNF/NC/SPCE was immersed in an activated Cas13a-crRNA complex solution containing 1.0×10⁶ fg/ml of S gene for various incubation times (0.25 hours to 4 hours). ΔI increased with increasing reaction time and saturated at 1.5 hours. Therefore, the trans-cleavage time of 1.5 hours was selected as the optimal time for SARS-CoV-2 detection.

### Example 5. Analytical Performance of Electrochemical Sensors

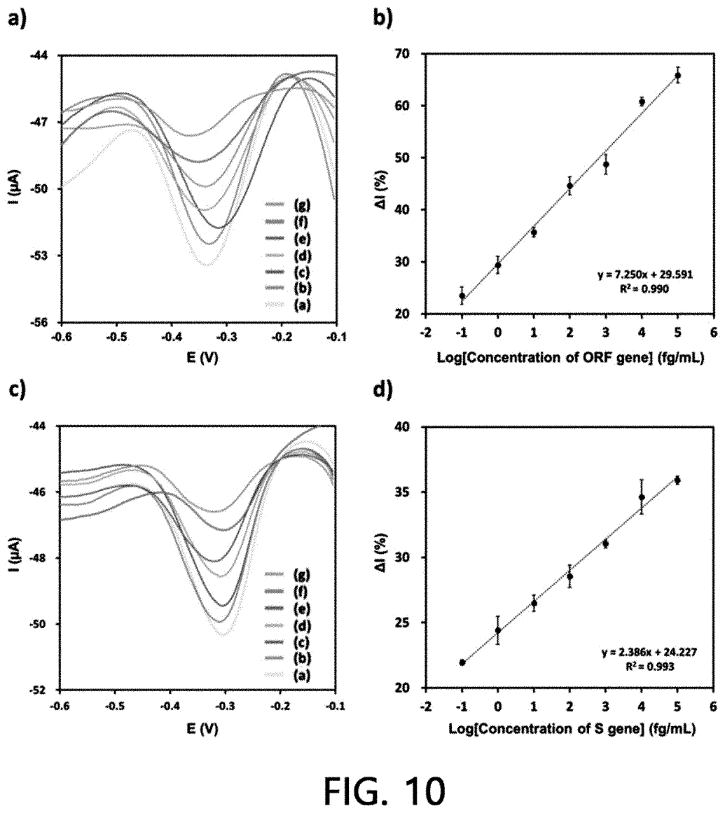

The analytical performance of the designed sensor was evaluated by performing DPV experiments on serial dilution ORF and S genes of SARS-CoV-2 in 0.1M PBS containing 0.1M KCl under the optimized experimental conditions. As can be seen from (a) and (c) in FIG. 10, the Faraday peak current obtained by DPV gradually decreased as the concentrations of ORF and S genes decreased. The reduction current was reduced due to the cleavage of MB-labeled reRNA by trans-cleavage activity from the Cas13a-crRNA complex. Calibration plots of ORF and S genes were obtained in the range of 1.0×10⁻¹ fg/ml to 1.0×10⁵ fg/ml ((b) and (d) in FIG. 10). The current change was linearly related to the logarithm of each gene concentration and mapped as a correlation of ΔI %=7.250×log C_{ORFgene} X+29.591 and ΔI %=2.386×log C_{S gene} X+24.227 (R²=0.995). The limit of detection (LOD) of the constructed sensor was estimated to be 4.4×10⁻² fg/ml of ORF and 8.1×10⁻² fg/ml of S gene, with respect to the current change of the blank sample and the sum of three standard deviations. All experiments were performed in triplicate at different concentrations. Considering that the LOD value of the sensor derived under the optimal condition is lower than the reported concentration of SARS-CoV-2 RNA in saliva (1.0×10³ fg/ml to 1.0×10⁷ fg/ml), for rapid detection of viral RNA, the trans-cleavage time was reduced to 30 minutes, and additional experiments were performed using a blank value and a minimum RNA concentration of saliva, 1.0×10³ fg/ml (Bar-On, et al., 2020. elife 9, e57309.; Zhu, et al., 2020. Journal of Infection 81(3), e48-e50). The blank values of ΔI % of the S gene and ORF gene were 17.06% and 19.22%, respectively, and 67.91% and 32.51% at a concentration of 1.0×10⁶ fg/ml (data not illustrated) were obtained, which indicates that the biosensor can be utilized for SARS-CoV-2 screening in a short period of time. Compared to other nucleic acid amplification-based detection methods, the sensor established by the present invention showed a surprising ability to detect a small amount of SARS-CoV-2 gene and a wide linear range without gene amplification technology.

In addition, the reproducibility of the biosensor was examined by evaluating intra- and inter-assay variability. The relative standard deviation (RSD) was evaluated 4 times using the ORF and S genes of 1.0×10³ fg/ml under the optimal conditions. RSDs of internal and internal analyzes were estimated to be 3.14% (n=4) and 2.52% (n=4) for the ORF gene and 2.47% (n=4) and 1.74% (n=4) for the S gene, respectively. From these results, it was possible to confirm the reliable reproducibility of the present invention.

### Example 6. On-Site Detection Applicability Test Using Artificial Salivary Spike SARS-CoV-2 RNA

The detection accuracy was verified whether it is possible to apply the biosensor for the detection of SARS-CoV-2 RNA in saliva samples. The amount of SARS-CoV-2 RNA in the saliva of patients was reported to be 1.0×10⁴ copies/ml to 1.0×10⁸ copies/ml. This copy number range can be converted to 1.0×10³ fg/ml to 1.0×10⁷ fg/ml. With respect to this concentration range of SARS-CoV-2 RNA, SARS-CoV-2 RNA was serially diluted to minimal and intermediate levels (1.0×10³ fg/ml and 1.0×10⁵ fg/ml) using artificial saliva, and was quantified under the optimal conditions with the developed biosensor. As shown in Table 2, the recoveries according to the ORF and S gene concentrations were 109.42% to 111.33% and 96.54% to 101.21%, respectively, within the allowable range. The recovery of the spiked sample was calculated by dividing the amount of SARS-CoV-2 RNA detected using the biosensor by the amount of SARS-CoV-2 RNA added to the artificial saliva sample. The above result shows that the biosensing system construed for SARS-CoV-2 RNA detection can be applied to the saliva sample matrix with high accuracy.

TABLE 2

| SARS-COV-2 | Spiked concentration | Detected concentration | Recovery |
|---|---|---|---|
| gene | (fg/ [symbol] ) | (fg/ [symbol] ) | (%) |
| ORF gene | 1.00 × 10³ | 1.09 ± 0.30 × 10³ | 109.42 |
| | 1.00 × 10⁵ | 1.11 ± 0.13 × 10⁵ | 111.33 |
| S gene | 1.00 × 10³ | 0.97 ± 0.12 × 10³ | 96.54 |
| | 1.00 × 10⁵ | 1.01 ± 0.17 × 10⁵ | 101.21 |

The present invention can detect a very small amount of target RNA with high sensitivity without a nucleic acid amplification reaction through the CRISPR/Cas13a trans-cleavage reaction. Thus, since the present invention can detect the target RNA with high accuracy while minimizing the test time, it has the advantage of being usefully used for on-site diagnostic test of RNA-based infectious diseases with fast spread.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

### DESCRIPTION OF REFERENCE NUMERALS

NC: nanocomposite
SPCE: screen-printed carbon electrodes
GNP: Graphene nanoplatelets
CHT: chitosan
MoS₂ NSs: Molybdenum disulfide nanosheets
AuNF: flower-shaped gold nanostructure
MPA: 3-mercaptopropionic acid

What is claimed is:

1. A biosensor for detecting a target RNA in which a reporter RNA (reRAN) is immobilized on an electrode on which a nanocomposite (NC) containing molybdenum disulfide (MoS₂), graphene, and chitosan (CHT) and a flower-shaped gold nanostructure (AuNF) are deposited.

2. The biosensor of claim 1, wherein the biosensor reacts with a Cas13a-crRNA-target RNA complex so that a current is reduced.

3. The biosensor of claim 1, wherein the biosensor is coated with a blocking agent.

4. The biosensor of claim 3, wherein the blocking agent is BSA, SKIM MILK, SALMON SPERM DNA, or mercaptohexanol (MCH).

5. The biosensor of claim 1, wherein the reporter RNA is immobilized on the electrode on which the nanocomposite and nanostructure are deposited through a streptavidin-biotin bond, an avidin-biotin bond, or a thiol-gold bond.

6. The biosensor of claim 1, wherein the nanocomposite contains the molybdenum disulfide, the graphene, and the chitosan in a volume ratio of 1:0.3 to 0.7:0.05 to 0.3.

7. The biosensor of claim 1, wherein the reporter RNA is tagged with a redox molecule.

8. The biosensor of claim 7, wherein the redox molecule is methylene blue, toluidine blue or ferrocene.

9. The biosensor of claim 1, wherein the electrode is a carbon electrode.

10. A method for manufacturing the biosensor of claim 1, comprising the steps of:
(a) sequentially depositing the nanocomposite containing the molybdenum disulfide (MoS₂), the graphene, and the chitosan and the flower-shaped gold nanostructure on the electrode; and
(b) immobilizing the reporter RNA (reRNA) tagged with a redox molecule to the electrode on which the nanocomposite and nanostructure are deposited.

11. The method of claim 10, wherein in the step (a), 3-mercaptopropionic acid (MPA) is treated on the electrode on which the nanocomposite and the nanostructure are sequentially deposited.

12. The method of claim 11, wherein in the step (a), 0.05 M to 0.5 M of the 3-mercaptopropionic acid is treated for 10 minutes to 1 hour.

13. The method of claim 11, wherein in the step (a), the 3-mercaptopropionic acid-treated electrode is coated with a blocking agent.

14. The method of claim 13, wherein the blocking agent is BSA, SKIM MILK, SALMON SPERM DNA, or mercaptohexanol (MCH).

15. The method of claim 10, wherein in the step (a), the electrode on which the nanocomposite and nanostructure are sequentially deposited is coated with streptavidin, avidin, or biotin.

16. The method of claim 15, wherein in the step (b), in order to interact with the streptavidin, avidin, or biotin coated on the gold on the electrode surface or the electrode surface in the step (a), the reporter RNA (reRNA) each bound to a biotin group, a streptavidin group, or a thiol group is reacted and immobilized.

17. The method of claim 15, wherein in the step (a), 1 mg/ml to 20 mg/ml of the streptavidin is added to be coated.

18. The method of claim 10, wherein in the step (b), a reaction with 50 μg/ml to 500 μg/ml of the reporter RNA is performed for 2 hours to 6 hours.

19. A method for detecting a target RNA using the biosensor of claim 1, comprising the steps of:
(a) treating a Cas13a-crRNA-RNA mixture sample on the biosensor of claim 1; and
(b) measuring a reduced current amount of the biosensor.

20. The method of claim 19, wherein the target RNA is a SARS-CoV-2 RNA, and the crRNA is the crRNA of an ORF gene represented by a nucleotide sequence of SEQ ID NO: 3 and/or a S gene represented by a nucleotide sequence of SEQ ID NO: 4.

21. The method of claim 19, wherein in the Cas13a-crRNA-RNA mixture sample, an RNA sample is additionally mixed with a mixture in which the Cas13a and the crRNA are mixed in a mass ratio of 1:0.1 to 0.001.

22. The method of claim 19, wherein in the step (a), the Cas13a-crRNA-RNA mixture sample is treated on the biosensor and reacted for 1 hour to 2 hours.

23. The method of claim 21, wherein the RNA sample is included in a biological sample selected from the group consisting of whole blood, plasma, serum, urine, saliva, runny nose, upper respiratory tract mucus, lower respiratory tract mucus, excretion, lymph, amniotic fluid, and tissue, or the RNA sample is selected from the biological sample.

24. A kit for detecting a target RNA comprising the biosensor of claim 1, Cas13a, and a target RNA-specific crRNA.

25. The kit of claim 24, wherein the target RNA is a SARS-CoV-2 RNA, and the crRNA is the crRNA of an ORF gene represented by a nucleotide sequence of SEQ ID NO: 3 and/or a S gene represented by a nucleotide sequence of SEQ ID NO: 4.
